(19) United States
(12) Patent Application Publication
(10) Pub. No.: US 2007/0161968 A1
(43) Pub. Date: Jul. 12, 2007

(54) Implantable medical device with pharmacologically active ingredient

(75) Inventors: Frank J. Fischer JR., Bloomington, IN (US); Jessica Watts Miller, Terre Haute, IN (US)

Correspondence Address: BRINKS HOFER GILSON & LIONE/CHICAGO/COOK, PO BOX 10395, CHICAGO, IL, 60610, US

(73) Assignee: Vance Products Inc., dba Cood Urological Inc., Spencer, IN (US)

(21) Appl. No.: 11/638,142

(22) Filed: Dec. 13, 2006

Related U.S. Application Data
(63) Continuation-in-part of application No. 10/410,587, filed on Apr. 8, 2003.
Application No. 10/410,587 is a continuation-in-part of application No. 08/868,518, filed on Jun. 4, 1997, now Pat. No. 6,599,275.
(60) Provisional application No. 60/018,924, filed on Jun. 4, 1996.

(51) Int. Cl. A61M 31/00

(52) U.S. Cl. 604/508; 604/265

(57) ABSTRACT

An implantable medical device comprising an inner region and an outer region positioned over at least a portion of the inner region and in contact with a surface of the inner region. The durometer of the inner region is greater than the durometer of the outer region and a pharmacologically active ingredient is present in at least a portion of the inner region or the outer region.

This application is a continuation-in-part of application Ser. No. 10/410,587, filed on Apr. 8, 2003, which is a continuation-in-part of application Ser. No. 08/868,518, filed on Jun. 4, 1997, now U.S. Pat. No. 6,599,275, and entitled “Implantable Medical Device”, which claims the benefit of provisional application Ser. No. 60/018,924, filed on Jun. 4, 1996. Each of these applications and patents is hereby incorporated by reference in its entirety.

### TECHNICAL FIELD

This invention relates generally to medical devices and, particularly, to medical devices that are implantable either partly or completely into a human or veterinary patient.

### BACKGROUND OF THE INVENTION

It has become common to treat a variety of medical conditions by introducing an implantable medical device partly or completely into the esophagus, trachea, colon, biliary tract, urinary tract, vascular system or other location within a human or veterinary patient. For example, many treatments of the vascular system entail the introduction of a device such as a stent, a catheter, a balloon, a wire guide, a cannula, or the like. However, when such a device is introduced into and manipulated through the vascular system, the blood vessel walls can be disturbed or injured. Clot formation or thrombosis often results at the injured site, causing stenosis or occlusion of the blood vessel. Moreover, if the medical device is left within the patient for an extended period of time, a thrombus often forms on the device itself, again causing stenosis or occlusion. As a result, the patient is placed at risk of a variety of complications, including heart attack, pulmonary embolism, and stroke. Thus, the use of such a medical device can entail the risk of precisely the problems that its use was intended to ameliorate.

Another problem associated with implantable medical devices and, more particularly, to partly implanted medical devices such as catheters percutaneously introduced into the vascular, or other, system of a patient for long-term hemodialysis or drug infusion is the risk of infection. This risk is also present with hyperalimentation (intravenous feeding) catheters which are percutaneously introduced into the patient. The urinary tract is also subjected to such risks when an urethral catheter, such as a Foley catheter, is introduced into the patient's bladder via the urethra for the drainage of urine.

In an attempt to reduce the risk of infection, a pharmacologically active ingredient, such as an antibiotic, has been used in conjunction with the catheter. Various coatings including antibiotics have been utilized in the past. However, the antibiotic typically is dispersed or dissipated from the coating in a relatively short period of time. Although effective in short-term implantation, such coatings are typically ineffective for extended duration placement such as with hemodialysis, drug infusion, or urinary tract catheters, which can be implanted in the patient for two to three years at a time.

### SUMMARY OF THE INVENTION

The foregoing problems are solved and a technical advance is achieved in an improvement to a medical device that is implantable either partly or completely into a human or veterinary patient. One aspect of the present invention provides an implantable medical device comprising an elongated inner region having an inner surface defining a lumen and an outer surface, and an elongated outer region positioned over at least a portion of the elongated inner region and in contact with the outer surface of the elongated inner region. The durometer of the elongated inner region is greater than the durometer of the elongated outer region and a releasable pharmacologically active ingredient is present in at least a portion of one of the elongated inner region and the elongated outer region.

In one embodiment, the elongated inner region includes a polymer, which may be a polyurethane. In another embodiment, the elongated outer region includes a polymer, which may be a silicone.

In another embodiment, a first pharmacologically active ingredient is present in at least a portion of the elongated inner region and a second pharmacologically active ingredient is present in at least a portion of the elongated outer region.

In yet another embodiment, the elongated inner region and the elongated outer region are coextruded.

In another embodiment, the pharmacologically active ingredient is selected from the group consisting of an anesthetic, an antiseptic, an antimicrobial agent, an antiviral agent, an antibiotic, an antiproliferative agent, an anti-cancer chemotherapeutic agent, an antithrombogenic agent and an antiflammatory agent.

In other embodiments the implantable medical device is an urinary catheter, an ureteral catheter or stent, a long term urinary device, an urethral catheter or stent, a prostatic stent, a biliary stent, a pancreatic stent, a catheter for suprapubic drainage, a catheter for nephrostomy drainage, a catheter for nasal pancreatic drainage, a nasal biliary drainage catheter, a tissue bonding urinary device, a penile prosthesis, a wound drain tube, a hydrocephalus shunt, a peritoneal catheter or an artificial urinary sphincter.

In yet another embodiment the implantable medical device includes an inner region having an inner surface and an outer surface, and an outer region positioned over at least a portion of the inner region and in contact with the outer surface of the inner region. The durometer of the inner region is greater than the durometer of the outer region. A pharmacologically active ingredient is present in at least a portion of one of the inner region and the outer region.

In another embodiment, the durometer of the inner region is between about 40 to 80 on the Shore D Hardness Scale and the durometer of the outer region is between about 30 to 90 on the Shore A Hardness Scale.

Another aspect of the present invention provides for a method of delivering a pharmaceutically active ingredient to a patient. In one embodiment, the method includes at least partially implanting a medical device within the patient. The medical device includes an inner region and an outer region positioned over at least a portion of the inner region and in contact with the inner region. The durometer of the inner region is greater than the durometer of the outer region. The pharmacologically active ingredient is present in at least a portion of one of the inner region and the outer region. The medical device is present within the patient for a time period sufficient to allow at least a portion of the pharmacologically active ingredient to be delivered to the patient.

### BRIEF DESCRIPTION OF THE DRAWINGS

FIG. 1 depicts a cross-sectioned end view of one embodiment of the implantable medical device of the present invention;

FIG. 2 depicts a cross-sectioned end view of another embodiment of the implantable medical device of the present invention;

FIG. 3 depicts a cross-sectioned end view of yet another embodiment of the implantable medical device of the present invention; and

FIG. 4 depicts a cross-sectioned end view of another embodiment of the implantable medical device of the present invention.

### DETAILED DESCRIPTION

Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

The terms “about” or “substantially” used with reference to a quantity includes variations in the recited quantity that are equivalent to the quantity recited, such as an amount that is insubstantially different from a recited quantity for an intended purpose or function.

As used herein, the term “implantable” refers to an ability of a medical device to be positioned, partially or wholly, at a location within a body of a human or veterinary patient for any suitable period of time, such as within a body vessel. For example, the medical device may be implanted within an esophagus, trachea, colon, biliary tract, urinary tract, or vascular system of a patient. Furthermore, the terms “implantation” and “implanted” refer to the positioning of a medical device, partially or wholly, at a location within a body, such as within a body vessel. Implantable medical devices can be configured for transient placement within a body vessel during a medical intervention (e.g., minutes to hours), or to remain in a body vessel for a prolonged period of time after an implantation procedure (e.g., weeks or months or years). Implantable medical devices can include devices configured for bioabsorption within a body during a prolonged period of time.

The term “biodegradable” refers to materials selected to dissipate upon implantation within a body, independent of which mechanisms by which dissipation can occur, such as dissolution, degradation, absorption and excretion. The actual choice of which type of materials to use may readily be made by one of ordinary skill in the art. Such materials are often referred to by different terms in the art, such as “bioresorbable,” “bioabsorbable,” or “biodegradable”, depending upon the mechanism by which the material dissipates. The prefix “bio” indicates that the erosion occurs under physiological conditions, as opposed to other erosion processes, caused for example, by high temperature, strong acids or bases, UV light or weather conditions.

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause an undesirably adverse, long-lived or escalating biological reaction or response. Such a response is distinguished from a mild, transient inflammation which typically accompanies surgery or implantation of foreign objects into a living organism.

As used herein, the phrase “controlled release” refers to the release of a material, such as a pharmaceutically active ingredient, at a predetermined rate. A controlled release may be characterized by a drug elution profile, which shows the measured rate that the material is removed from a material-containing device in a given solvent environment as a function of time. A controlled release does not preclude an initial burst release associated with the deployment of the medical device. In some embodiments of the invention an initial burst, followed by a more gradual subsequent release, may be desirable. The release may be a gradient release in which the concentration of the material released varies over time or a steady state release in which the material is released in equal amounts over a certain period of time (with or without an initial burst release).

As used herein, the term durometer refers to a measure of the hardness of a material, for example, the material forming a portion of an implantable medical device. Durometer scales run from 0 to 100, with larger numbers indicating harder material. The different scales are identified by letters, including A, B, C and D. A standard test for the measurement of the durometer of materials such as plastics and elastomers is described in American Society for Testing and Materials (ASTM) D2240-05.

As used herein, the term pharmaceutically active ingredient refers to any agent that produces an intended therapeutic effect on the body to treat or prevent conditions or diseases.

As used herein, a “mixture” refers to a combination of two or more substances in which each substance retains its own chemical identity and properties.

Implantable Medical Device Having a Dual Durometer Portion

One aspect of the present invention provides an implantable medical device having and inner region and an outer region positioned over and in contact with the inner region and forming at least a portion of the surface of the medical device. The durometer of the inner region is greater than the durometer of the outer region. A pharmaceutically active ingredient is incorporated into at least one of the inner region or the outer region.

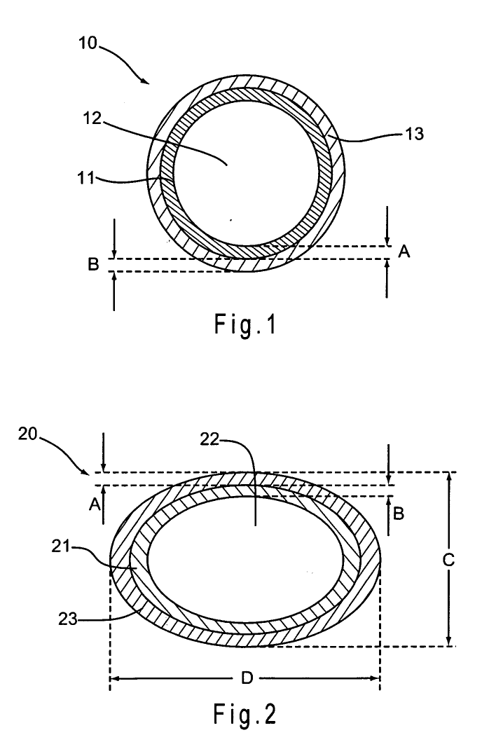

In one illustrative embodiment, the implantable medical device is a catheter or a similar device having a hollow elongated portion. FIG. 1 depicts a cross-sectioned end view of such an embodiment. Implantable medical device 10 includes hollow inner elongated region 11 with passage 12 extending longitudinally therein. Outer elongated region 13 is positioned over and in contact with region 11. In certain embodiments, outer elongated region 13 is positioned substantially coaxially with region 11.

In various embodiments, hollow elongated region 11 and outer elongated region 13 have a substantially circular cross section and the ratio of the radial thickness “A” of portion 11 to the radial thickness “B” of outer elongated region 13 is less than 10:1, 9:1, 8:1, 7:1, 6:1, 5:1, 4:1, 3:1, 2:1, 1:1, 0.9, 0.8:1, 0.7:1, 0.6:1, 0.5:1, 0.4:1, 0.3:1, 0.2:1or 0.1:1. In various embodiments, outer elongated region 13 has a substantially circular cross section having a diameter between 1 fr and 15 fr, 1 fr and 10 fr, 1 fr and 8 fr, 1 fr and 6 fr, 1 fr and 4 fr, 2 fr and 15 fr, 2 fr and 10 fr, 2 fr and 8 fr, 2 fr and 6 fr, 2 fr and 4 fr or 10 fr and 6 fr.

FIG. 2 depicts a cross-sectioned end view of another illustrative embodiment of an implantable medical device of the present invention. Medical device 20 includes hollow elongated region 21 with passage 22 extending longitudinally therein. Outer elongated region 23 is positioned over and in contact with region 21. In this embodiment, hollow elongated region 21 and outer elongated region 23 have a substantially elliptical cross section where the ratio of the maximum diameter “D” of outer region 23 to the minimum diameter “C” of outer region 23 is between 1 and 1.1, 1.2, 1.3, 1.4, 1.5, 1.7, 2.0 or 3.0. In various embodiments, and the ratio of the radial thickness “A” of portion 21 to the radial thickness “B” of portion 23 is less than 10:1, 9:1,8:1, 7:1, 6:1, 5:1, 4:1, 3:1, 2:1, 1:1, 0.9, 0.8:1, 0.7:1, 0.6:1, 0.5:1, 0.4:1, 0.3:1, 0.2:1 or 0.1:1. In various embodiments, outer elongated region 23 has a maximum diameter between1 fr and 15 fr, 1 fr and 10 fr, 1 fr and 8 fr, 1 fr and 6 fr, 1 fr and fr, 2 fr and 15 fr, 2 fr and 10 fr, 2 fr and 8 fr, 2 fr and 6 fr, 2 fr and 4 fr or 10 fr and 6 fr.

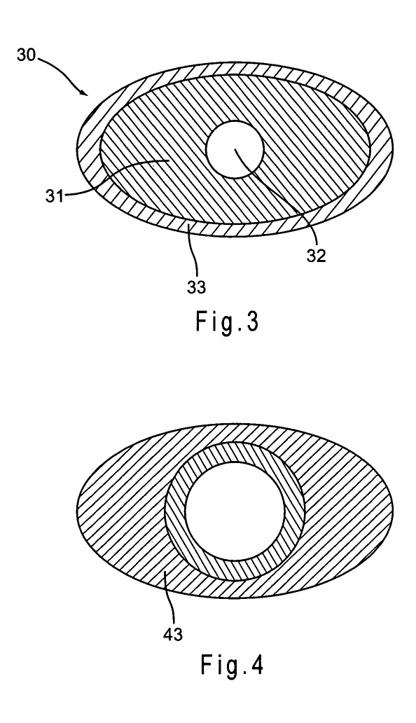

FIGS. 3 and 4 depict cross-sectioned end views of yet other illustrative embodiments of an implantable medical device of the present invention. In FIG. 3, the radial thickness of inner elongated region 31 varies, while in FIG. 4, the radial thickness of outer elongated region 43 varies. Of course, the present invention includes embodiments where the radial thickness of both the inner elongated region and the outer elongated region vary. The present invention also includes embodiments there the cross section of the inner elongated region and/or the outer elongated region is of another shape, such as rectangular, square, triangular, hexagonal or irregular.

One another embodiment, the inner region and outer region form the surface of an inflatable device, for example, a balloon such as that forming part of a Foley catheter. In yet another embodiment, the inner region and outer region form the two outermost layers of an implantable medical device such as a metallic stent. In such an embodiment, the inner region may be a layer covering a portion of the metallic surface of the stent and the outer region a second layer covering a portion of the inner region.

Composition of the Dual Durometer Portion of the Medical Device

A construction including an outer region having a durometer that is less than that of the inner region provides a medical device having a soft or flexible outer layer that is less likely to damage the wall of the body cavity into which it is implanted and a more rigid inner layer (for example, the inner elongated region) that provides increased tensile strength and rigidity to the device. In one embodiment, the durometer of the outer region is in a range of about 30 to 90 on the Shore A Hardness Scale. In other embodiments, the durometer of the outer region is in a range of about 40 to 90, 50 to 90, 60 to 90, 70 to 90, 80 to 90, 40 to 80, 40 to 70, 40 to 60 or 40 to 50 on the Shore A Hardness Scale. In another embodiment, the durometer of the inner region is in a range of 40 to 80 on the Shore D Hardness Scale. In other embodiments, the durometer of the inner region is in a range of 50 to 80, 60 to 80, 70 to 80, 40 to 70, 40 to 60 or 40 to 50 on the Shore D Hardness Scale.

In one embodiment of the present invention, the inner region includes a polymer, preferably a biocompatible polymer. In various illustrative embodiments, the inner region includes carbon, carbon fiber, cellulose acetate, cellulose nitrate, polyethylene teraphthalate, polyurethane, polyamide, polyester, polyorthoester, polyanhydride, polyether sulfone, polycarbonate, polypropylene, high molecular weight polyethylene, polytetrafluoroethylene, polylactic acid, polyglycolic acid, a polyanhydride, a silicone, a nylon, a natural or synthetic rubber, polyvinylchloride, polycaprolactone, polyhydroxybutyrate valerate, or another biocompatible polymeric material, or mixtures or copolymers of these materials. In a preferred embodiment, the inner region includes a polyurethane.

The outer region may also include the lower durometer grades of the polymers listed as suitable for use in the inner region. In one embodiment, a plasticizer is added to obtain the required durometer. Preferred polymers include a silicone or a poly(siloxane). For example, a silicone material having a durometer in a range of 30 to 90 on the Shore A Hardness Scale is commercially available from the NU-SIL Corporation of Carpinteria, Calif.

Besides the required durometer of the implantable medical device, selection of the appropriate material for the inner region or the outer region may depend upon the desired rate of release of a specific pharmaceutical ingredient (discussed below). The inner region and/or the outer region may also include additives, such as the pharmaceutically active ingredients discussed below as well as plasticizers, excipients, stabilizers or the like. In certain embodiments, the inner region and/or the outer region includes a biodegradable material, such as a biodegradable polymer.

In one embodiment, the outer region is formed over a preexisting inner region. Alternatively, the inner region and the outer region may be formed at the same time using a coextrusion process in which the two materials are extruded through a single die with two orifices arranged so that the extrudates merge and weld together into a single structure before solidifying. Some materials are not amenable to such coextrusion, for example, those having significantly different melting points and viscosity characteristics. Such process limitations are well known in the art.

Incorporation of a Pharmaceutically Ingredient

The implantable medical device of the present invention includes a pharmaceutically active ingredient incorporated into the inner region and/or the outer region. Various embodiments allow for the controlled release of the pharmaceutically active ingredient upon implantation of the device into a patient. By allowing for the delayed release of the pharmaceutically active ingredient when the medical device is implanted, such medical devices allow for amounts of the pharmaceutically active ingredient to be released for longer periods of time as compared to the release from previous devices. In various embodiments of the invention, less than 90 percent of the pharmaceutically active ingredient present in the medical device is released into a physiological environment over a period of at least about two years, one year, 6 months, two months, one month, one week, one day, 6 hours, 4 hours, 2 hours, 1 hr, 30 minutes or 15 minutes.

In various embodiments, the release of the pharmacologically active ingredient upon implantation is affected by factors including, but not limited to, the chemical structure of the pharmacologically active ingredient, the thickness of the inner or outer layers, the position of the pharmacologically active ingredient within the inner and/or outer layers, the material present in the inner and or the outer layers and the conditions used to incorporate the pharmacologically active ingredient into the inner and/or outer layers. For example, in one embodiment, the inner and/or outer layers include a polymeric material and the release of the pharmacologically active ingredient is dependent upon the nature of the polymer.

Preferably, the pharmacologically active ingredient includes one or more drugs, agents, or medicaments for concomitantly minimizing or treating the infection or affliction. Examples of pharmacologically active ingredients useful for inclusion in the implantable medical devices of the present can be found in U.S. Pat. No. 6,599,275, the contents of which are incorporated by reference. It is intended that the term pharmacologically active ingredient includes any material that is molecularly interactive with the fluids, cells, proteins or tissues of a human or an animal to augment the diagnosis, treatment or prevention of any physiologic or pathologic condition. It is further intended that this term includes therapeutic and diagnostic agents such as, for example, drugs, vaccines, hormones, steroids, proteins, previously described agents, complexing agents, salts, chemical compounds, polymers, and the like.

In certain embodiments, the pharmaceutically active ingredient is an anesthetic, an antiseptic, an antimicrobial agent, an antiviral agent, an antibiotic, an antiproliferative agent, an anti-cancer chemotherapeutic agent, antithrombogenic agent or an antiflammatory agent.

In other embodiments, the pharmaceutically active ingredient is an anesthetic such as lidocaine, bupivacaine or ropivacaine.

In yet other embodiments, the pharmaceutically active ingredient is an antimicrobial agent, for example, an antibacterial agent such as rifampin or minocycline; an antiseptic, such as chlorhexidine; an antibiotic, such as ampicillin, gentamicin or tobramycin, or an anti-fungal drug, such as amphotericin.

In other embodiments, the pharmaceutically active ingredient is a drug which prevents or ameliorates abrupt closure and restenosis of blood vessels previously opened by stenting surgery or other procedures.

Thrombolytics (which dissolve, break up or disperse thrombi) and antithrombogenics (which interfere with or prevent the formation of thrombi) are especially useful when the implantable medical device is inserted into the vascular system. Particularly preferred thrombolytics are urokinase, streptokinase, and the tissue plasminogen activators. Particularly preferred antithrombogenics are heparin, hirudin, and the antiplatelets.

Heparin is a mucopolysaccharide anticoagulant typically obtained from porcine intestinal mucosa or bovine lung. Heparin acts as a thrombin inhibitor by greatly enhancing the effects of the blood's endogenous antithrombin III. Thrombin, a potent enzyme in the coagulation cascade, is key in catalyzing the formation of fibrin. Therefore, by inhibiting thrombin, heparin inhibits the formation of fibrin thrombi. In certain embodiments, heparin may be covalently bound to the outer layer of the implantable medical device. Thus, heparin would form the outermost layer of the implantable medical device and would not be readily degraded enzymatically, and would remain active as a thrombin inhibitor.

Of course, pharmaceutically active ingredients having other functions can also be successfully delivered by the device of the present invention. For example, an antiproliferative agent such as methotrexate will inhibit over-proliferation of smooth muscle cells and thus inhibit restenosis of the dilated segment of the blood vessel. The antiproliferative is desirably supplied for this purpose over a period of about four to six months. Additionally, localized delivery of an antiproliferative agent is also useful for the treatment of a variety of malignant conditions characterized by highly vascular growth. In such cases, the medical device of the present invention could be placed in the arterial supply of the tumor to provide a means of delivering a relatively high dose of the antiproliferative agent directly to the tumor.

A vasodilator, such as a calcium channel blocker or a nitrate, will suppress vasospasm following angioplasty procedures. Vasospasm occurs as a response to injury of a blood vessel, and the tendency toward vasospasm decreases as the vessel heals. Accordingly, the vasodilator is desirably supplied over a period of about two to three weeks. Of course, trauma from angioplasty is not the only vessel injury which can cause vasospasm, and the medical device may be introduced into vessels other than the coronary arteries, such as the aorta, carotid arteries, renal arteries, iliac arteries or peripheral arteries for the prevention of vasospasm in them.

A variety of other pharmacologically active ingredients are particularly suitable for use when the implantable medical device is configured other than as a coronary stent. For example, an anti-cancer chemotherapeutic agent can be delivered by the device to a localized tumor. More particularly, the device can be placed in an artery supplying blood to the tumor or elsewhere to deliver a relatively high and prolonged dose of the agent directly to the tumor, while limiting systemic exposure and toxicity. The agent may be a curative, a pre-operative debulker reducing the size of the tumor, or a palliative which eases the symptoms of the disease. The pharmacologically active ingredient of the present invention may, of course, be released from the medical device into any lumen defined in the device, or to tissue in contact with the device and that the lumen may carry some other agent to be delivered through it. For example, drugs such as tamoxifen citrate, TAXOL® (Paclitaxel) or derivatives thereof PROSCAR® (Finasteride), HYTRIN® (Terazosin), or EULEXIN® (flutamide) may be incorporated into the implantable medical device and/or applied to a tissue-exposed surface of the device for delivery to a tumor located, for example in breast tissue or the prostate.

Dopamine or a dopamine agonist such as bromocriptine mesylate or pergolide mesylate is useful for the treatment of neurological disorders such as Parkinson's disease. The medical device could be placed in the vascular supply of the thalamic substantia nigra for this purpose, or elsewhere, localizing treatment in the thalamus.

A wide range of other pharmaceutically active ingredients can be delivered by the implantable medical device of the present invention. Accordingly, it is preferred that the pharmaceutically active ingredient contained in or posited on the inner region or the outer region includes at least one of heparin, covalent heparin, or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; HYTRIN® or other antihypertensive agent; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodeling inhibitor; deoxyribonucleic acid, an antisense nucleotide or another agent for molecular genetic intervention; methotrexate or another antimetabolite or antiproliferative agent; tamoxifen citrate, TAXOL® or the derivatives thereof, or other anti-cancer chemotherapeutic agents; dexamethasone, dexamethasone sodium phosphate, dexamethasone acetate or another dexamethasone derivative, or another anti-inflammatory steroid or non-steroidal antiinflammatory agent; cyclosporin, tacrolimus (FK-506), sirolimus (rapamycin), tacrolimus, everolimus, azathioprine, mycophenolate mofetil or another immunosuppressive agent; a mTOR inhibitor; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}Tc (6 hours) or another radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³¹-, ³²p- or ³⁶S-radiolabelled form or other radiolabelled form of any of the foregoing; estrogen or another sex hormone; AZT or other antipolymerases; acyclovir, famciclovir, rimantadine hydrochloride, ganciclovir sodium, NORVIR® (ritonavir), CRIXIVAN® (indinavir sulfate), or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; PROSCAR®, HYTRIN® or other agents for treating benign prostatic hyperplasia (BHP) or a mixture of any of these; various forms of small intestine submucosa (SIS); anesthetics such as lidocaine, bupivacaine or ropivacaine; or an antimicrobial agent, for example, an antibacterial agent such as rifampin or minocycline; an antiseptic, such as chlorhexidine; an antibiotic, such as ampicillin, gentamicin or tobramycin, or an anti-fungal drug, such as amphotericin.

Other pharmacologically active ingredients include additional drugs that are effective against urinary encrustation, in addition to heparin and other drugs listed above. These additional anti-encrustation drugs include triclosan, silver nitrate, ofloxacin, ciproflaxin, phosphorylcholine and trimethoprim. There are also additional drugs useful against microbes, including a penicillin, a cephalosporin, a carbepenem, a beta-lactam, an antibiotic, an aminoglycoside, a macrolide, a lincosamide, a glycopeptide, a tetracyline, a chloramphenicol, a quinolone, a fucidin, a sulfonamide, a trimethoprim, a rifamycin, an oxaline, a streptogramin, a lipopeptide, a ketolide, a polyene, an azole, and an echinocandin. Still other useful antimicrobial drugs with which an implantable medical device may be coated include alpha-terpineol, methylisothiazolone, cetylpyridinium chloride, chloroxyleneol, hexachlorophene, chlorhexidine and other cationic biguanides, methylene chloride, iodine and iodophores, triclosan, taurinamides, nitrofurantoin, methenamine, aldehydes, azylic acid, rifampycin, silver, benzyl peroxide, alcohols, and carboxylic acids and salts, and silver sulfadiazine. Also useful as antimicrobials are anthracyclines, such as doxorubicin or mitoxantrone, fluoropyrimidines such as 5-fluoroacil, and also podophylotoxins, such as etoposide. The salts and the derivatives of all of these are meant to be included as examples of antimicrobial drugs. Gendine, a mixture of chlorhexidine and Gentian Violet, is another useful antimicrobial drug.

Anticancer drugs may be useful to patients when placed into medical devices for at least partial insertion into a patient. These include docetaxel and its derivatives, fluoro-pyrimidines including 5-fluoroacil and its derivatives, hydroxyurea, mercaptopurine, cisplatin, anthracyclines including daunorubicin and doxorubicin and their derivatives, podophylotoxins including etoposide, and mitoxantrone and its derivatives, a folic acid antagonist other than methotrexate and its derivatives, a camptothecin, and a platinum complex.

Other pharmacologically active ingredients may also be used in embodiments. Alpha-blockers are drugs that block receptors in arteries and smooth muscles. The action of the alpha-blocker relaxes blood vessels and leads to an increase in blood flow and a lower blood pressure, thus helping to control blood pressure or hypertension. In the bladder neck or urinary tract, alpha-blockers also relax the walls of the tract and enhance urinary flow, especially in persons suffering from prostatic hypertrophy (an enlarged prostate gland). Alpha-blocker drugs include doxazosin (CARDURA®), alfuzosin (UROXATRAL®), tamsulosin (FLOMAX)®, prazosin (MINIPRESS®), and terazosin (HYTRIN®).

Calcium channel blockers (CCBs) are drugs that block the entry of calcium into muscle cells. By blocking the entry of calcium, the contraction of the heart is decreased and the arteries are dilated, reducing pressure in the arteries and making blood flow easier. Calcium channel blockers that may be used in medical device embodiments include nisoldipine (SULAR®), nifedipine (ADALAT®, PROCARDIA®), nicardipine (CARDENE®), bepridil (VASCOR®), isradipine (DYNACIRC®), nimodipine (NIMOTOP®), felodipine (PLENDIL®), amlodipine (NORVASC®), diltiazem (CARDIZEM®) and verapamil (CALAN®, ISOPTIN®).

Other pharmacologically active ingredients that are useful in a human or mammalian body include analgesics and anesthetics. In general, an anesthetic works by interrupting the transmission of nerve impulses, and thus preventing the sensation of pain. Analgesics work on the peripheral and central nervous systems to reduce the perception of pain. Aspirin was the first analgesic. Analgesics include naproxen, choline, diflunisal, and salsalate. Other analgesics include non-steroidal antiflammatory agents, such as naproxen, choline, diflunisal, salsalate, fenoprofen, flurbiprofen, ketoprofen, ibuprofen, oxaprozin, diclofenac, indomethacin, sulindac, acetoaminophen, tolmetin, meloxicam, piroxicam, meclofenamate, mefanimic acid, nabumetone, etodelac, keterolac, celecoxib, valdecoxib and rofecoxib, mixtures thereof, and derivatives thereof.

Other analgesics include opioids, synthetic drugs with narcotic properties, and narcotics such as alfentanil, buprenorphine, carfentanil, codeine, codeinone, dextropropoxyphene, dihydrocodeine, endorphin, -fentanyl, hydrocodone, hydromorphone, methadone, morphine, morphinone, oxycodone, oxymorphone, pethidine, remifantanil, sulfentanil, thebaine, and tramadol, mixtures thereof, and derivatives thereof. Anesthetics which may be used as a pharmacologically active ingredient in medical devices for implantation include local anesthetics such as paracetamol, bupivacaine, prilocaine, levobupivicaine, dubucaine, ropivacaine, lidocaine, and novocaine.

Metals, especially heavy metals, and ionic compounds and salts of these metals, are known to be useful as antimicrobials even in very low amounts or concentrations. These substances are said to have an oligodynamic effect, and they are considered oligodynamic. The metals include silver, gold, zinc, copper, cerium, gallium, platinum, palladium, rhodium, iridium, ruthenium, osmium, zinc, bismuth, and others. Other metals with lower atomic weights also have an inhibiting or cidal effect on microorganisms in very low concentrations. These metals include aluminum, calcium, sodium, magnesium, potassium, manganese, and lithium, among others. For present purposes all these metals are oligodynamic metals, and their compounds and ionic substances are oligodynamic substances. The metals, their compounds and ions, e.g., zinc oxide, silver acetate, silver nitrate, silver chloride, silver iodide and many others, may inhibit the growth of microorganisms, such as bacteria, viruses, or fungi, or they may have cidal effects on microorganisms, such as bacteria, viruses, or fungi, in higher concentrations. Because many of these compounds and salts are soluble, they may easily be placed into solution, alone or with another physiologically active ingredient, and then absorbed into a medical device or adsorbed onto its surface.

The anionic portion of the compound or salt is desirably selected from among, but is not limited to, the oxide, acetate, ascorbate, benzoate, bitartrate, bromide, carbonate, chloride, citrate, folate, gluconate, iodate, iodide, lactate, laurate, oxalate, palmitate, perborate, phenosulfonate, phosphate, propionate, salicylate, stearate, succinate, sulfadiazine, sulfate, sulfide, sulfonate, tartrate, thiocyanate, thioglycolate, thiosulfate, and the like. Combinations of any of these may also be used.

Silver salts are particularly useful for their inhibiting and cidal effects on microorganisms, such as bacteria, viruses and fungi. Such salts include, but are not limited to, silver oxide, silver chloride, silver iodide, silver citrate, silver lactate, silver acetate, silver propionate, silver salicylate, silver bromide, silver ascorbate, silver laurel sulfate, silver phosphate, silver sulfate, silver benzoate, silver carbonate, silver sulfadiazine, silver gluconate, and combinations thereof.

Oligodynamic substances as defined above, including the metals, the salts, and other compounds, may advantageously be used in combination with other physiologically active ingredients. The salts and compounds may be particularly useful because, having different solubilities, the appropriate salts or compounds may be selected for the desired rate of release within the patient. The compound or compounds with the desired inhibiting or cidal effect and the desired release may be selected in coordination with another medicament or drug for the desired effect on the patient.

It is to be understood, however, that the above-described implantable medical device is merely an illustrative embodiment of the principles of this invention, and that other devices and methods for using them may be devised by those skilled in the art, without departing from the spirit and scope of the invention. It is to be understood that the invention is directed to embodiments both comprising and consisting of the disclosed parts. It is contemplated that only parts of the device can include the pharmacologically active ingredient. Furthermore, different parts of the device can include different pharmacologically active ingredients. It is also contemplated that different sides or regions of the same part of the medical device can include different pharmacologically active ingredients.

Incorporation of a Pharmaceutically Active Ingredient into the Implantable Medical Device

In one embodiment, the implantable medical device comprises at least one pharmaceutically active ingredient within the material forming the inner region and/or the outer region. The inner region may contain the same or a different pharmaceutically active ingredient to that present in the outer region. Alternatively, a pharmaceutically active ingredient may be present in only one of the outer or the inner region. In this manner, one or more pharmaceutically active ingredients may be delivered, for example, with a vascular stent or catheter, to the blood stream from the lumen surface of the stent, and a different treatment may be delivered on the vessel surface of the stent.

In one embodiment, a first pharmaceutically active ingredient with a higher diffusion rate than a second pharmaceutically active ingredient is present in the inner region while the second pharmaceutically active ingredient, with a lower diffusion rate is present in the outer region. In this embodiment, the ingredient having the lower diffusion rate is closer to the outer surface of the device, enabling the ingredients to achieve the objective of reaching the “outer surfaces” of the device at a predetermined relative timing, including substantially simultaneously.

In one such embodiment, the medical device is a catheter and the pharmacologically active ingredient includes a 50:50 mixture by weight of minocycline and rifampin. Minocycline has a lower diffusion rate than rifampin and, as a result, is included in the material of outer region. The minocycline is also included in the inner region.

Of course, other materials, including other pharmaceutically active ingredients, can be located at other locations in or on the implantable medical device, including on the surfaces of the outer and/or inner regions.

In one embodiment, a mixture of a polymer and a pharmaceutically active ingredient is extruded or coextruded to form at least a portion of the implantable medical device. Such a method of manufacture is suitable for those pharmaceutically active ingredients that a stable under the conditions, particularly the temperature, required for the extrusion process.

For example, in one embodiment, a powered base silicone material is mixed with the pharmacologically active ingredient in a solvent. The mixture is then extruded at low temperatures with the solvent evaporating as the silicone material cures. Low temperature silicone is utilized so as not to evaporate or inactivate the pharmacologically active ingredient.

In another embodiment, a pharmaceutically active ingredient is incorporated into the material of the inner and/or the outer region after the formation of the implantable medical device. U.S. Pat. No. 5,624,704, which is hereby incorporated by reference, teaches methods of incorporating pharmaceutically active ingredients into the material of an implantable medical device.

In one embodiment, one or more pharmacologically active ingredients, such as analgesics or anesthetics, may be impregnated into such devices by contacting at least a portion of the device with a mixture of the pharmaceutically active ingredient, a solvent and a penetrating substance. The solvent is preferably an organic solvent. The penetrating agent is a substance that enables the pharmacologically active ingredient to permeate the base material or layers of the device intended for implantation. In certain other embodiments, a penetrating agent is not required to achieve impregnation of the pharmacologically active ingredient.

The organic solvent can be any solvent that can be used to dissolve pharmacologically active ingredient. For example, alcohols (e.g. methanol, ethanol), ketones (e.g. acetone, methylethylketone), ethers (e.g. tetrahydrofuran), aldehydes (e.g. formaldehyde), acetonitrile, acetic acid, methylene chloride, chloroform, and other organic solvents.

The penetrating agent can be any organic compound that can be used to promote penetration of the pharmacologically active ingredient into the material of the medical device. Examples of these compounds are esters (e.g. ethyl acetate, propyl acetate, butyl acetate, amyl acetate, and combinations thereof), ketones (e.g. acetone and methylethylketone), methylene chloride, chloroform, and other suitable solvents.

Examples of alkalinizing agents include an organic or inorganic base including sodium hydroxide, potassium hydroxide, ammonia in water (27% ammonium hydroxide), diethylamine and triethylamine. The high ionic strength salt can be any salt exhibiting high ionic strength, such as sodium chloride, potassium chloride and ammonium acetate. These salts may act both as an alkalinizing agent and as a penetrating agent to enhance the receptivity of the medical device material.

One embodiment of the present invention provides a method for impregnating a non-metallic portion of a medical device with a pharmacologically active ingredient. The method comprises the steps of forming a pharmacologically active ingredient of an effective concentration by dissolving the ingredient in an organic solvent, adding a penetrating agent to the composition and applying the ingredient to at least a portion of medical device under conditions where the pharmacologically active ingredient permeates the material of the medical device and is positioned within the device.

Another embodiment of the present invention is a method for impregnating a non-metallic portion of a medical device with a pharmacologically active ingredient comprising the steps of forming a pharmacologically active ingredient of an effective concentration by dissolving the ingredient in an organic solvent, and applying the ingredient to at least a portion of medical device under conditions where the pharmacologically active ingredient permeates the material of the medical device and is positioned within the device. In certain embodiments, the organic solvent is methanol or ethanol. In another embodiment, the pharmacologically active ingredient is bupivacaine.

In one embodiment, it is desirable to impregnate a medical device, such as a stent or a catheter for implantation, in only part of the device. For instance, only the portion of a urethral or Foley catheter near or protruding through the urethral meatus may need to be coated with a pharmaceutically active ingredient. If a patient is being treated for cancer only in a ureter, a ureteral stent coated only in the central linear portion may be appropriate, rather than also coating the portions of the stent that will lie in the kidney or the bladder. Limiting the impregnated portion of the implanted medical device to that portion where the drug or medicament is needed at least has the effect of reducing the amount of drug absorbed by the patient. This may help in reducing over-medicating of patients and minimize any adverse drug reactions, and may also reduce any sensitization effects.

Medical devices may be impregnated in only part of their structures by masking those portions or otherwise preventing impregnation of a pharmaceutically active ingredient into the medical device. The masking may be accomplished physically, as by limiting the travel of medical devices that are dipped into solution. The surfaces may also be blocked or masked physically or chemically. One way is to coat or mask the areas into which impregnation of the pharmacologically active ingredient is not desired with a coating that is easily removed at the end of the impregnation process. In this way, only those portions of the device requiring impregnation are contacted with the pharmaceutically active ingredient. Contacting may occur through dipping, soaking, spraying, or other method for impregnating the pharmacologically active ingredient into the medical device.

Of course, as is mentioned above, in some cases a penetrating agent is not required to achieve impregnation of the pharmacologically active ingredient into the device.

Exemplary Implantable Medical Devices

Exemplary medical devices having a dual durometer portion and that are amenable to the incorporation of pharmacologically active ingredients, by impregnation or otherwise, generally include a non-metallic material, such as one of the polymeric materials described above. However, where materials having differing durometers are to be coextruded together, care must be taken to ensure that compatible materials are chosen.

Medical devices according to the present invention preferably include a portion that is fixed in size, having a constant cross-section, rather than being expandable in one or more dimensions. Examples of such device include a double-pigtail ureteral stent or a urinary Foley catheter. These medical devices may have some variability in their inner diameter or outer diameter, i.e., in the sense that no device has perfect dimensional stability, and also in the sense that some parts of the device may be larger than other parts. When a ureteral stent is implanted into a ureter, or when a Foley catheter is implanted into a bladder and a urethra, there may be some compression of the walls of the stent or catheter, leading to a minor “change” in the inner diameter or outer diameter of the device. However, except for the balloon protion of the Foley catheter, these devices are not “radially expandable.” These devices are thus not similar to a vascular stent in which there is an intentional and desired change in the radial dimension so that the stent may be implanted and expanded to fulfill its intended purpose in a blood vessel. Implantable medical devices having a constant cross section include the urinary catheters and ureteral stents as described above, which include a majority portion with a constant cross section that does not change in size upon implantation. Medical devices, as the term is used herein, do not include vascular stents whose cross section and dimensions change abruptly upon implantation into a patient.

Particular medical devices especially suited for application of materials with differing durometers having pharmacologically active ingredients incorporated according to this invention include urinary catheters, for example a Foley catheter, ureteral catheters or stents, prostatic stents, long term urinary devices, urethral catheters or stents, biliary stents, pancreatic stents, catheters for suprapubic drainage, catheters for nephrostomy drainage, catheters for nasal pancreatic drainage, nasal biliary drainage catheters, tissue bonding urinary devices, penile prostheses, wound drain tubes, hydrocephalus shunts, peritoneal catheters, artificial urinary sphincters, and the like.

Methods of Treating a Patient

Another aspect of the present invention provides for a method of administrating a pharmaceutically active ingredient to a human or veterinary patient. In one embodiment, the method comprises implanting a medical device of the present invention into the body of the patient. Such a device contains the pharmaceutically active ingredient within the inner region and/or the outer region. In one embodiment, the implantable medical device is a urethral or ureteral catheter or stent, for example, a Foley catheter introduced into the patient's bladder via the urethra for the drainage of urine. In another embodiment, an antimicrobial agent, such as chlorhexidine, is incorporated into such a device to reduce the risk of infection. In yet another embodiment, an anesthetic agent, such as bupivacaine, is incorporated into the device to reduce or eliminate pain associated with the placement of the device. In certain embodiments, two or more pharmaceutically active ingredients are incorporated into the device, either within the same region of the device or within separate regions. For example, one pharmaceutically active ingredient is incorporated within the inner region and another pharmaceutically active ingredient is incorporated within the outer region.

### EXAMPLE 1

Method for Impregnating a Non-Metallic Medical Implant with a Pharmacologically Active Ingredient

One embodiment of the present invention is a method for impregnating a non-metallic medical device with a pharmacologically active ingredient. The method includes forming a pharmacologically active ingredient at an effective concentration by dissolving the pharmacologically active ingredient in an organic solvent, adding a penetrating agent to the composition, and applying the composition to at least a portion of medical device under conditions where the pharmacologically active ingredient permeates the material of the medical device.

In a preferred embodiment, the step of dissolving a pharmacologically active ingredient may also include the step of adding an alkalinizing agent to the composition. Further according to one embodiment, the pharmacologically active ingredient is heated to a temperature of between about 30° C. and 70° C. prior to applying the composition to the medical device to increase the adherence of the pharmacologically active ingredient to the material of the medical device. After the impregnated device is removed from the solution of the pharmacologically active ingredient and allowed to dry, the impregnated device is preferably rinsed with a liquid and milked to remove excess granular deposits and ensure uniform color of the impregnated device. The pharmacologically active ingredient may be applied to the medical device by dipping the implant into a solution of the dissolved ingredient for a period of between 15 and 120 minutes. Preferably, the device is dipped in the composition for a period of approximately 60 minutes.

In one aspect, a method of the present invention preferably comprises a single step of applying a pharmacologically active ingredient to the surfaces of a medical device. However, it is expected that several applications of the pharmacologically active ingredient, or other substance, can be applied to the surfaces of the device without affecting the adherence of the pharmacologically active ingredient to the device.

It is to be understood, that the above-described implantable medical device is merely an illustrative embodiment of the principles of this invention, and that other devices and methods for using them may be devised by those skilled in the art, without departing from the spirit and scope of the invention. It is to be understood that the invention is directed to embodiments both comprising and consisting of the disclosed parts. It is contemplated that only parts of the device can include the pharmacologically active ingredient. Furthermore, different parts of the device can include different pharmacologically active ingredients. It is also contemplated that different sides or regions of the same part of the device can include different pharmacologically active ingredients or layers. Accordingly, the invention should be limited only by the spirit and scope of the claims.

We claim:

1. An implantable medical device, comprising:
an elongated inner region having an inner surface defining a lumen and an outer surface;
an elongated outer region positioned over at least a portion of the elongated inner region and in contact with the outer surface of the elongated inner region, wherein the durometer of the elongated inner region is greater than the durometer of the elongated outer region,
wherein a pharmacologically active ingredient is present in at least a portion of one of the elongated inner region and the elongated outer region.

2. The implantable medical device of claim 1, wherein the elongated inner region comprises a polymer.

3. The implantable medical device of claim 2, wherein the polymer comprises a polyurethane.

4. The implantable medical device of claim 1, where the elongated outer region comprises a polymer.

5. The implantable medical device of claim 4, where the polymer comprises a silicone.

6. The implantable medical device of claim 1, wherein the elongated inner region comprises a polyurethane and the elongated outer region comprises a silicone.

7. The implantable medical device of claim 1, wherein the pharmacologically active ingredient is present in at least a portion of the elongated outer region.

8. The implantable medical device of claim 1, wherein the pharmacologically active ingredient is present in at least a portion of the elongated inner region.

9. The implantable medical device of claim 1, wherein a first pharmacologically active ingredient is present in at least a portion of the elongated inner region and a second pharmacologically active ingredient is present in at least a portion of the elongated outer region.

10. The implantable medical device of claim 1, wherein the elongated outer region is extruded over the elongated inner region.

11. The implantable medical device of claim 1, wherein the elongated inner region and the elongated outer region are coextruded.

12. The implantable medical device of claim 1, wherein the pharmaceutically active ingredient is selected from the group consisting of an anesthetic, an antiseptic, an antimicrobial agent, an antiviral agent, an antibiotic, an antiproliferative agent, an anti-cancer chemotherapeutic agent, an antithrombogenic agent and an antiflammatory agent.

13. The implantable medical device of claim 1, wherein the pharmaceutically active ingredient is an anesthetic.

14. The implantable medical device of claim 14, wherein the anesthetic is bupivacaine.

15. The implantable medical device of claim 1, wherein the pharmaceutically active ingredient is an antiseptic.

16. The implantable medical device of claim 15, wherein the antiseptic is chlorhexidine.

17. The implantable medical device of claim 1, wherein the implantable medical device is selected from the group consisting of an urinary catheter, an ureteral catheter or stent, a long term urinary device, an urethral catheter or stent, a prostatic stent, a biliary stent, a pancreatic stent, a catheter for suprapubic drainage, a catheter for nephrostomy drainage, a catheter for nasal pancreatic drainage, a nasal biliary drainage catheter, a tissue bonding urinary device, a penile prosthesis, a wound drain tube, a hydrocephalus shunt, a peritoneal catheter and an artificial urinary sphincter.

18. The implantable medical device of claim 1, wherein the durometer of the elongated inner region is between about 40 to 80 on the Shore D Hardness Scale and the durometer of the elongated outer region is between about 30 to 90 on the Shore A Hardness Scale.

19. An implantable medical device, comprising:
an inner region having an inner surface and an outer surface; and
an outer region positioned over at least a portion of the inner region and in contact with the outer surface of the inner region,
wherein the durometer of the inner region is greater than the durometer of the outer region and wherein a pharmacologically active ingredient is present in at least a portion of one of the inner region and the outer region.

20. A method of delivering a pharmaceutically active ingredient to a patient, the method comprising:
at least partially implanting a medical device within the patient, the medically device comprising an inner region having an inner surface and an outer surface and an outer region positioned over at least a portion of the inner region and in contact with the outer surface of the inner region, wherein the durometer of the inner region is greater than the durometer of the outer region and wherein the pharmacologically active ingredient is present in at least a portion of one of the inner region and the outer region,
wherein the medical device is present within the patient for a time period sufficient to allow at least a portion of the pharmacologically active ingredient to be delivered to the patient.
